(19) United States
(12) Patent Application Publication
(10) Pub. No.: US 2013/0178350 A1
(43) Pub. Date: Jul. 11, 2013

(54) METHOD FOR MANUFACTURING DISPOSABLE WORN ARTICLE

(71) Applicant: Toyoshi Umebayashi, Osaka (JP)

(72) Inventor: Toyoshi Umebayashi, Osaka (JP)

(73) Assignee: ZUIKO CORPORATION, Osaka (JP)

(21) Appl. No.: 13/825,619

(22) PCT Filed: Nov. 8, 2011
(86) PCT No.: PCT/JP2011/075692

§ 371 Date: Mar. 22, 2013

(30) Foreign Application Priority Data
Nov. 18, 2010 (JP) 2010-257631

(51) Int. Cl. A61F 13/15

(52) U.S. Cl. CPC A61F 13/15756
USPC 493/382

(57) ABSTRACT

A method for manufacturing a disposable worn article including an absorbent body 2 and an external sheet 3, the method including the steps of: carrying a continuous sheet in a carrying direction X, wherein the continuous sheet includes external sheets 3 continuous with one another in the carrying direction, the external sheets 3 being stretchable in a girth direction Y and substantially less stretchable in the carrying direction X perpendicular to the girth direction Y; placing the absorbent body 2 on the continuous sheet while a front portion 2f, a crotch portion 2c and a rear portion 2b are lined up in the carrying direction X; and severing the continuous sheet along a virtual cut-off line extending in the girth direction Y, thereby obtaining individual worn articles each having the absorbent body 2 placed on the external sheet 3.

### TECHNICAL FIELD

The present invention relates to a method for manufacturing a disposable worn article.

### BACKGROUND ART

A method for manufacturing a disposable worn article while carrying a sheet and a core in the longitudinal direction perpendicular to the girth direction has been proposed in the art (see the first patent document). In the manufacturing method disclosed in the first patent document, identified below, liquid-absorbing cores are placed intermittently between a continuous top sheet and a continuous back sheet, thereby forming a laminate, after which the laminate is cut into individual disposable worn articles.

The second patent document, identified below, also discloses an external sheet obtained by laminating together a stretchable web that can be stretched in the girth direction and a non-stretchable web. However, it fails to give any disclosure as to the stretchability in the longitudinal direction. It also fails to give any disclosure as to the manufacturing method.

### CITATION LIST

### Patent Literature

[First Patent Document] Japanese Laid-Open Patent Publication No. 1-162808 (FIG. 4)
[Second Patent Document] Japanese Laid-Open Patent Publication No. 10-99373 (Paragraphs 0008, 0009)

### SUMMARY OF INVENTION

While a tension is given in the carrying direction of the continuous sheet to be the external sheet, the tension causes the continuous sheet to stretch in the carrying direction. This results in necking, where the continuous sheet shrinks in the girth direction perpendicular to the carrying direction, causing the finishing precision (accuracy of finishing) of the worn article to lower.

The amount of stretch in the carrying direction varies along with variation in the tension. Therefore, misalignment easily occurs to the position of the leg holes and the absorbent body with respect to the continuous sheet, causing the finishing precision to lower.

Therefore, it is an object of the present invention to provide a method for manufacturing a disposable worn article, with which the cause for lowering the finishing precision of a worn article is reduced.

A first aspect of the present invention is directed to a method for manufacturing a disposable worn article including an absorbent body and an external sheet, the absorbent body including a liquid-permeable top sheet forming a skin-contact surface to be in contact with a wearer, a liquid-impermeable back sheet forming a non-skin-contact surface on an opposite side of the skin-contact surface, and a core sandwiched between the top sheet and the back sheet for absorbing body fluid, the absorbent body including a front portion, a crotch portion and a rear portion covering a front surface of a torso of the wearer, a crotch of the wearer and a rear surface of the torso of the wearer, respectively, and the external sheet being fastened to the back sheet of the absorbent body and projecting, in a girth direction of the wearer, from the absorbent body in the front portion and the rear portion of the absorbent body, the method including the steps of:

carrying, in a carrying direction perpendicular to the girth direction, a continuous sheet to be the external sheet that is stretchable in the girth direction and substantially less stretchable in the carrying direction, while the continuous sheet is continuous in the carrying direction;

placing the absorbent body on the continuous sheet while the front portion, the crotch portion and the rear portion are lined up in the carrying direction; and

severing the continuous sheet along a virtual cut-off line extending in the girth direction, thereby obtaining individual worn articles each having the absorbent body placed on the external sheet.

A second aspect of the present invention is directed to a method for manufacturing a disposable worn article including an absorbent body and an external sheet, the absorbent body including a liquid-permeable top sheet forming a skin-contact surface to be in contact with a wearer, a liquid-impermeable back sheet forming a non-skin-contact surface on an opposite side of the skin-contact surface, and a core sandwiched between the top sheet and the back sheet for absorbing body fluid, the absorbent body including a front portion, a crotch portion and a rear portion covering a front surface of a torso of the wearer, a crotch of the wearer and a rear surface of the torso of the wearer, respectively, and the external sheet being fastened to the back sheet of the absorbent body and projecting, in a girth direction of the wearer, from the absorbent body in the front portion and the rear portion of the absorbent body, the method including the steps of:

carrying, in a carrying direction perpendicular to the girth direction, a continuous sheet to be the external sheet of the worn article that is stretchable in the girth direction and substantially less stretchable in the carrying direction, while the continuous sheet is continuous in the carrying direction;

successively cutting off portions to be the external sheets from a tip portion of the continuous sheet in the carrying direction; and

placing the absorbent body so that the absorbent body spans (bridges) between the cut-off portions to be the external sheets.

In the present invention, since an external sheet that is substantially less stretchable in the carrying direction is used, the external sheet is unlikely to stretch in the carrying direction while carrying the external sheet. Therefore, necking described above is unlikely to occur, or positional misalignment is unlikely to occur for leg holes and absorbent bodies.

In the present invention, there may be an object interposed between the external sheet fastened to the back sheet and the back sheet.

### BRIEF DESCRIPTION OF DRAWINGS

FIG. 1A is a plan view showing a worn article, unfolded, according to Embodiment 1 of the present invention, and FIG. 1B is a cross-sectional view taken along line IB-IB in FIG. 1A.

FIGS. 2A and 2B are plan views each showing Embodiment 1 of the manufacturing method of the present invention.

FIG. 3A is a plan view showing a worn article, unfolded, according to Embodiment 2 of the present invention, and FIG. 3B is a cross-sectional view taken along line IIIB-IIIB in FIG. 3A.

FIG. 4 is a schematic plan/perspective view showing Embodiment 2 of the manufacturing method of the present invention.

FIG. 5A is a plan view showing a worn article, unfolded, according to Embodiment 3 of the present invention, and FIG. 5B is a cross-sectional view taken along line VB-VB in FIG. 5A.

FIGS. 6A and 6B are plan views each showing Embodiment 3 of the manufacturing method of the present invention.

FIG. 7A is a plan view showing a worn article, unfolded, according to Embodiment 4 of the present invention, and FIG. 7B is a cross-sectional view taken along line VIIB-VIIB in FIG. 7A.

FIG. 8 is a schematic plan/perspective view showing Embodiment 4 of the manufacturing method of the present invention.

### DESCRIPTION OF EMBODIMENTS

In a preferred embodiment in the first aspect of the present invention, the method further includes the steps of: carrying a continuous laminate to be a plurality of absorbent bodies in the carrying direction while the continuous laminate is continuous in the carrying direction; successively cutting off the absorbent bodies from a tip portion of the continuous laminate in the carrying direction; and separating the cut-off absorbent bodies from one another in the carrying direction of the absorbent bodies, wherein: in the placement step, the absorbent bodies are placed intermittently on the continuous sheet; and the continuous sheet is severed between the adjacent absorbent bodies.

In this case, since absorbent bodies are placed intermittently, the external sheet is stretchable in the girth direction in the girth area between the absorbent bodies. This improves the fit to the wearer.

In a preferred embodiment in the second aspect of the present invention, each portion to be the external sheet includes a first side portion projecting in the girth direction from the front portion of one external sheet and a second side portion projecting in the girth direction from the rear portion of another external sheet, the second side portion being continuous with the first side portion; and the method further includes the step of successively dividing the portion to be the external sheet along a virtual cut-off line extending in the girth direction between the first side portion and the second side portion in the portion to be the external sheet, and obtaining individual worn articles each having the absorbent body placed between the two divided external sheets.

In this case, portions to be external sheets are continuous with each other via an absorbent body therebetween, thus improving the handling.

On the other hand, the portions to be external sheets do not cover the entire absorbent body, but cover only a part of the absorbent body. Therefore, there is unlikely to be a waste of material.

In a more preferred embodiment in the first and second aspects, in the step of carrying the continuous sheet, no tensile force in the girth direction is given to the continuous sheet.

In this case, necking is even more unlikely to occur in the continuous sheet.

In another preferred embodiment of the present invention, an elastic band for giving stretchability in the girth direction to a rear waist portion covering the rear surface of the torso of the wearer is placed on the rear waist portion of the external sheet, and an inner surface of the external sheet is exposed so as to be in contact with a skin of the wearer between the elastic band and the absorbent body; and

the method further includes the step of intermittently placing a plurality of elastic bands on the continuous sheet while no tensile force is given in the girth direction of the elastic bands.

In this case, because of the elastic band provided in the rear waist portion, the disposable worn article fits around the waist, thus improving the wearability. Between the elastic band and the absorbent body, the inner surface of the external sheet is exposed so as to be in contact with the skin. Therefore, the air permeability in the rear waist portion is maintained.

Since the elastic band is placed on the rear waist portion while no tensile force is given to the elastic band, no tensile force in the girth direction is given to the continuous sheet. Therefore, necking is unlikely to occur in the continuous sheet.

With an external sheet used in the present invention, typically, the stress when stretched by 10% in the longitudinal direction is 10 times or more that when stretched by 10% in the girth direction. A method for manufacturing a non-woven fabric forming such an external sheet is disclosed in Japanese Laid-Open Patent Publication No. 9-279453, the entire disclosure of which is herein incorporated by reference.

The present invention will be understood more clearly from the following description of preferred embodiments taken in conjunction with the accompanying drawings. Note however that the embodiments and the drawings are merely illustrative and should not be taken to define the scope of the present invention.

The scope of the present invention shall be defined only by the appended claims. In the accompanying drawings, like reference numerals denote like components throughout the plurality of figures.

### Embodiment 1

Embodiments of the present invention will now be described with reference to the drawings.

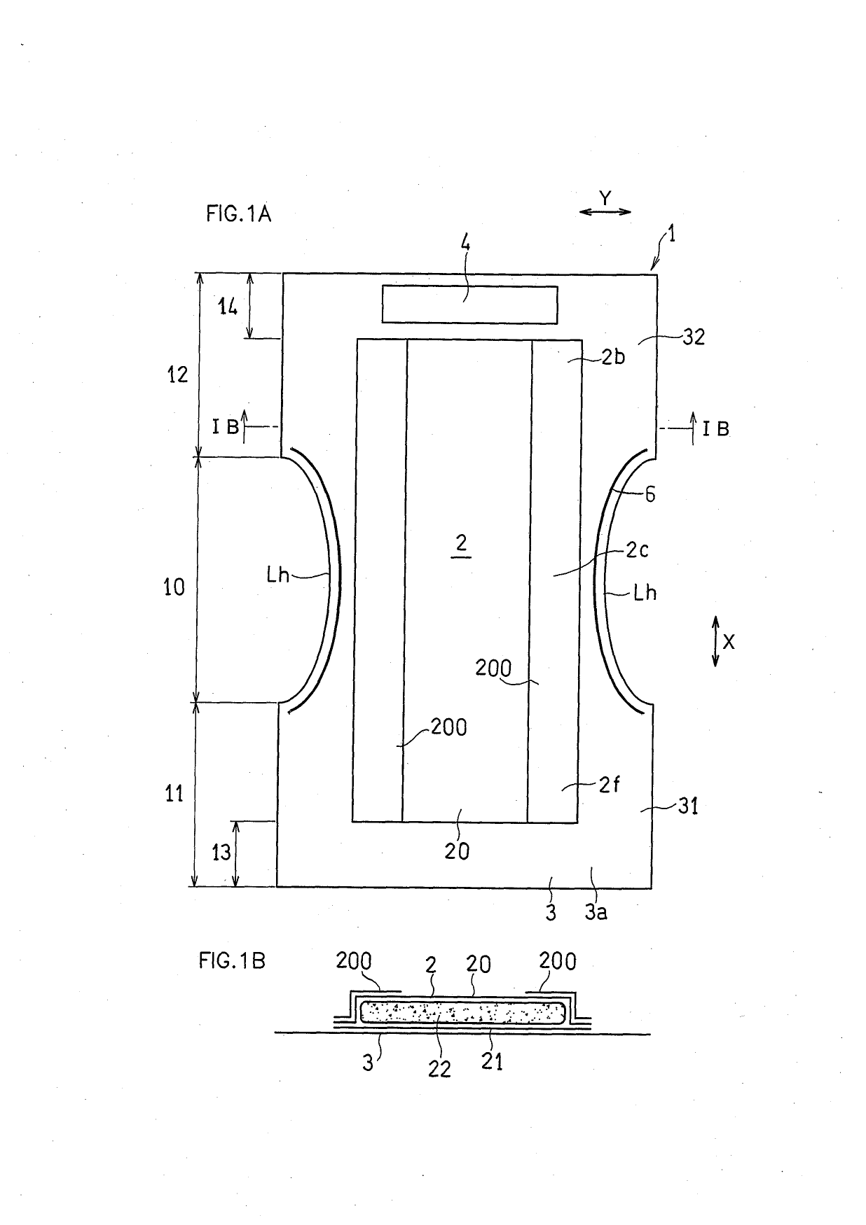

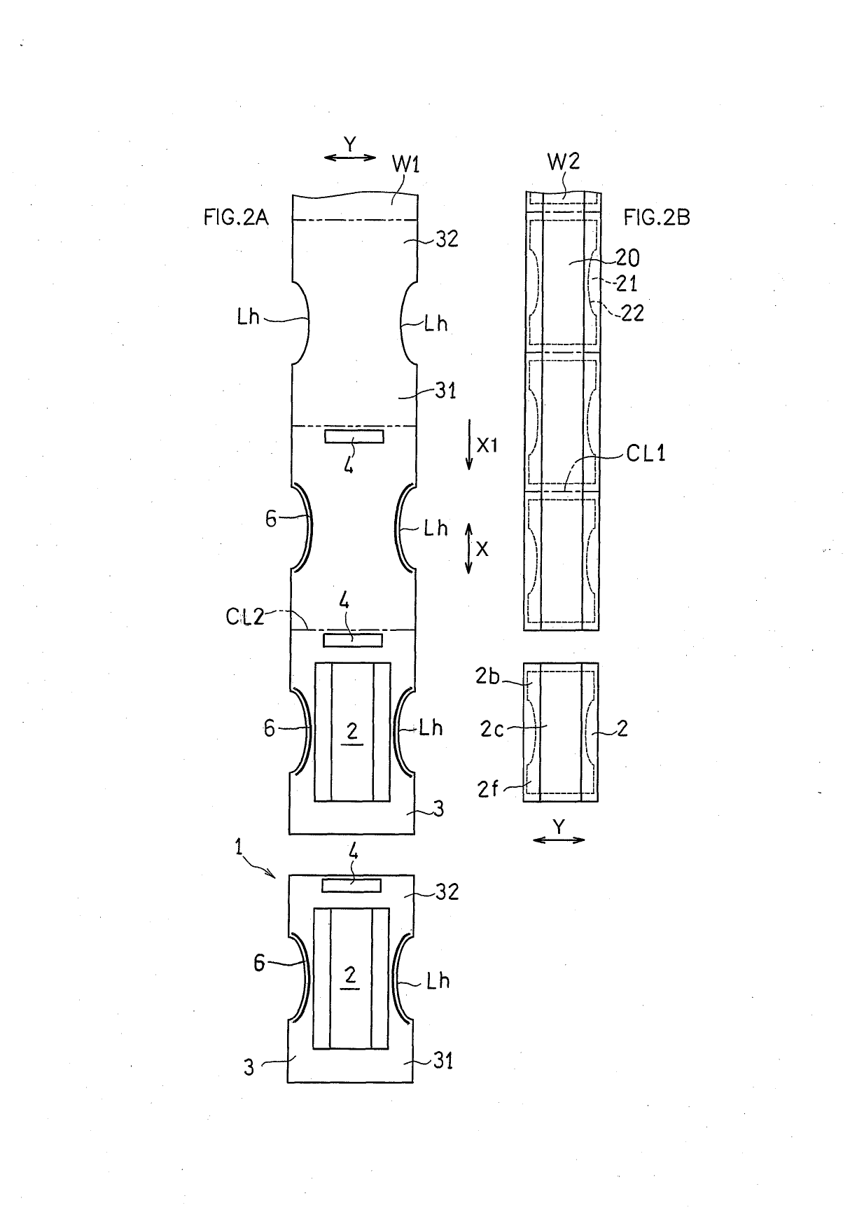

FIGS. 1A to 2B show Embodiment 1.

Diaper 1:

A diaper (an example of a worn article) 1 shown in FIG. 1A is, for example, a pants-type diaper, and includes a crotch portion 10 covering the crotch of the wearer, a front torso portion 11 covering the abdominal side of the wearer, and a rear torso portion 12 covering the back side of the wearer.

The diaper 1 includes an absorbent body 2 and an external sheet 3. The absorbent body 2 includes a front portion 2f, a crotch portion 2c and a rear portion 2b covering the front surface of the torso of the wearer, the crotch of the wearer, and the rear surface of the torso of the wearer, respectively.

The external sheet 3 is formed by an air-permeable material (e.g., non-woven fabric). The external sheet 3 is formed by an anisotropic non-woven fabric that is stretchable in the girth direction Y and substantially less stretchable in the longitudinal direction X perpendicular to the girth direction Y.

As shown in FIG. 1B, the absorbent body 2 includes a liquid-permeable top sheet 20 and a liquid-impermeable back sheet 21, with a core 22 for absorbing body fluid sandwiched therebetween.

The external sheet 3 is fastened to the back sheet 21 of the absorbent body 2, and is projecting in the girth direction of the wearer from the absorbent body 2 in the front portion 2f and the rear portion 2b of the absorbent body 2.

The core 22 is made of, for example, a pulp, a high-absorbent polymer, or the like.

A hydrophilic non-woven fabric may be placed as the core 22. The top sheet 20 is made of a non-woven fabric covering the inner surface of the core 22, forming a skin-contact surface to be in contact with the wearer, and the top sheet 20 may be provided with a three-dimensional non-woven fabric (cuffs) 200 for preventing side leaks. The back sheet 21 forms a non-skin-contact surface on the opposite side of the skin-contact surface, and covers the outer surface of the core 22.

While the absorbent body 2 is placed on an inner surface 3a of the external sheet 3, as shown in FIG. 1A, it may be detachably fastened to the external sheet 3 via a touch fastener (not shown) therebetween.

A front waist portion 13 and a rear waist portion 14, which are not covered by the absorbent body 2, are formed in the front torso portion 11 and the rear torso portion 12 of the external sheet 3, respectively.

The waist portions 13 and 14 of the external sheet 3 are not covered by the absorbent body 2. Therefore, if a material having good air permeability (e.g., a non-woven fabric, or the like) is employed as the external sheet 3, the air permeability of the waist portions 13 and 14 is improved, thereby preventing dampness.

The top sheet 20 and the back sheet 21 form the absorbent body 2 whose area is smaller than the external sheet 3. Thus, the area of the top sheet 20 and the back sheet 21 is significantly reduced. Thus, there is no waste of material.

An elastic band 4 for giving stretchability in the girth direction Y to the rear waist portion 14 may be placed on the rear waist portion 14, which covers the back side of the wearer. For example, a plurality of rubber threads, a rubber tape, a film, a material including a thermoplastic resin, or the like, may be employed as the elastic band 4.

Therefore, when the diaper 1 is worn, the inner surface 3a of the external sheet 3, made of a non-woven fabric, is exposed so as to be in contact with the skin of the wearer in the front waist portion 13. On the other hand, the inner surface 3a of the external sheet 3 is in contact with the skin of the wearer between the elastic band 4 of the rear waist portion 14 and the absorbent body 2.

In this embodiment, because of the elastic band 4 provided in the rear waist portion 14 in addition to the shrinking force from the external sheet 3, the disposable worn article fits around the waist, and the pants will not slip down when worn, thus improving the wearability. Here, between the elastic band 4 and the absorbent body 2, the inner surface of the external sheet 3 is exposed so as to be in contact with the skin. Therefore, the air permeability in the rear waist portion 14 is maintained.

A first side portion 31 projecting in the girth direction Y from the front portion 2f of the external sheet 3 and a second side portion 32 projecting in the girth direction Y from the rear portion 2b of the external sheet 3 are welded together, forming the diaper 1 into a pants shape. Cut-outs to be leg holes Lh, through which legs of the wearer are passed, are formed between the first side portion 31 and the second side portion 32.

In the vicinity of the opposite side edges in the girth direction Y of the absorbent body 2 corresponding to the leg holes Lh of the external sheet 3, elastic members 6 made of rubber threads, for example, may be placed so that the external sheet 3 conforms around the legs of the wearer.

When the diaper 1 is worn, the diaper 1 fits around the waist of the wearer because of the shrinkage of the stretched elastic band 4 in addition to the shrinkage of the external sheet 3. On the other hand, the elastic members 6 placed in the vicinity of the leg holes Lh shrink, whereby the diaper 1 fits around the legs of the wearer.

Note that the worn article may be a tape-type or fastener-type diaper, or the like, in which fastening members are provided so that the first side portion 31 and the second side portion 32 are not welded together but are fastened to each other by the fastening members.

Next, a method for manufacturing the diaper 1 will be described.

As shown in FIG. 2A, a continuous sheet W1 made of an anisotropic non-woven fabric is carried in the carrying direction X1. The continuous sheet W1 includes a plurality of external sheets 3 of FIG. 1A continuous with one another in the longitudinal direction X perpendicular to the girth direction Y. Therefore, the continuous sheet W1 is stretchable in the girth direction Y and substantially less stretchable in the carrying direction X1 perpendicular to the girth direction Y.

While the external sheet 3 and the continuous sheet W1 are carried, a tensile force is given in the longitudinal direction X and the carrying direction X1, but no tensile force is given in the girth direction Y. That is, the continuous sheet W1 is carried while being unstretched and shrunk in the girth direction Y.

Therefore, the external sheet 3 and the continuous sheet W1 are essentially unstretchable either in the carrying direction X1 (the longitudinal direction X) or the girth direction Y, thus avoiding necking.

Trimming Step:

While being carried, a portion of the continuous sheet W1 is trimmed off in a die-cutting process so that a portion of the external sheet 3 is narrowed between the first and second side edges 31 and 32, thus forming cut-outs to be the leg holes Lh.

After the trimming, the elastic bands 4 stretchable at least in the girth direction Y are placed with predetermined intervals. In this process, the elastic bands 4 are placed intermittently on the continuous sheet W1 while no tensile force is given in the girth direction Y of the elastic band 4, i.e., the elastic bands 4 are placed while being shrunk in the girth direction Y.

Step of Cutting Off Absorbent Body 2:

On the other hand, as shown in FIG. 2B, a continuous laminate W2 is carried in the carrying direction X1. The continuous laminate W2 includes the absorbent bodies 2 of FIG. 1 continuous with one another in the longitudinal direction X perpendicular to the girth direction Y.

The continuous laminate W2 includes the cores 22 sandwiched between the continuous top sheet 20 and the continuous back sheet 21. As shown by broken lines of FIG. 2B, the cores 22 are placed intermittently between the top sheet 20 and the back sheet 21.

The continuous laminate W2 is successively cut, into the absorbent bodies 2, along the first cut-off line CL1 generally parallel to the girth direction Y at the tip portion of the continuous laminate W2 in the carrying direction X1.

Separation Step:

After the severing (cutting off), as shown in FIG. 2B, the cut-off absorbent bodies 2 are separated from one another in the longitudinal direction X. For example, a re-pitching drum disclosed in Japanese Laid-Open Patent Publication No. 2006-212307 may be employed as the method for separating the absorbent bodies 2.

Step of Placing Absorbent Body 2:

As shown in FIG. 2A, after the elastic members 6 made of rubber threads, for example, are intermittently placed on the continuous sheet W1 along the longitudinal direction X in the vicinity of the leg holes Lh, the absorbent bodies 2 are intermittently placed on the continuous sheet W1.

In this process, the absorbent bodies 2 of FIG. 2B are placed on the continuous sheet W1 while the front portion 2f, the crotch portion 2c and the rear portion 2b are lined up in the carrying direction X1. That is, the absorbent bodies 2 are placed on the continuous sheet W1 so that the longitudinal direction X of the absorbent bodies 2 coincides with the longitudinal direction X of the external sheet 3.

Step of Obtaining Diaper 1:

Then, as shown in FIG. 2A, the continuous sheet W1 is severed along the second cut-off line CL2, which is generally parallel to the girth direction Y. In this process, the continuous sheet W1 is severed along the second cut-off line CL2 between adjacent absorbent bodies 2. Thus, individual diapers 1 are obtained, each including the absorbent body 2 placed on the external sheet 3.

Note that the trimming step may be performed after the step of placing the absorbent bodies 2 or the step of obtaining individual diapers 1.

### Embodiment 2

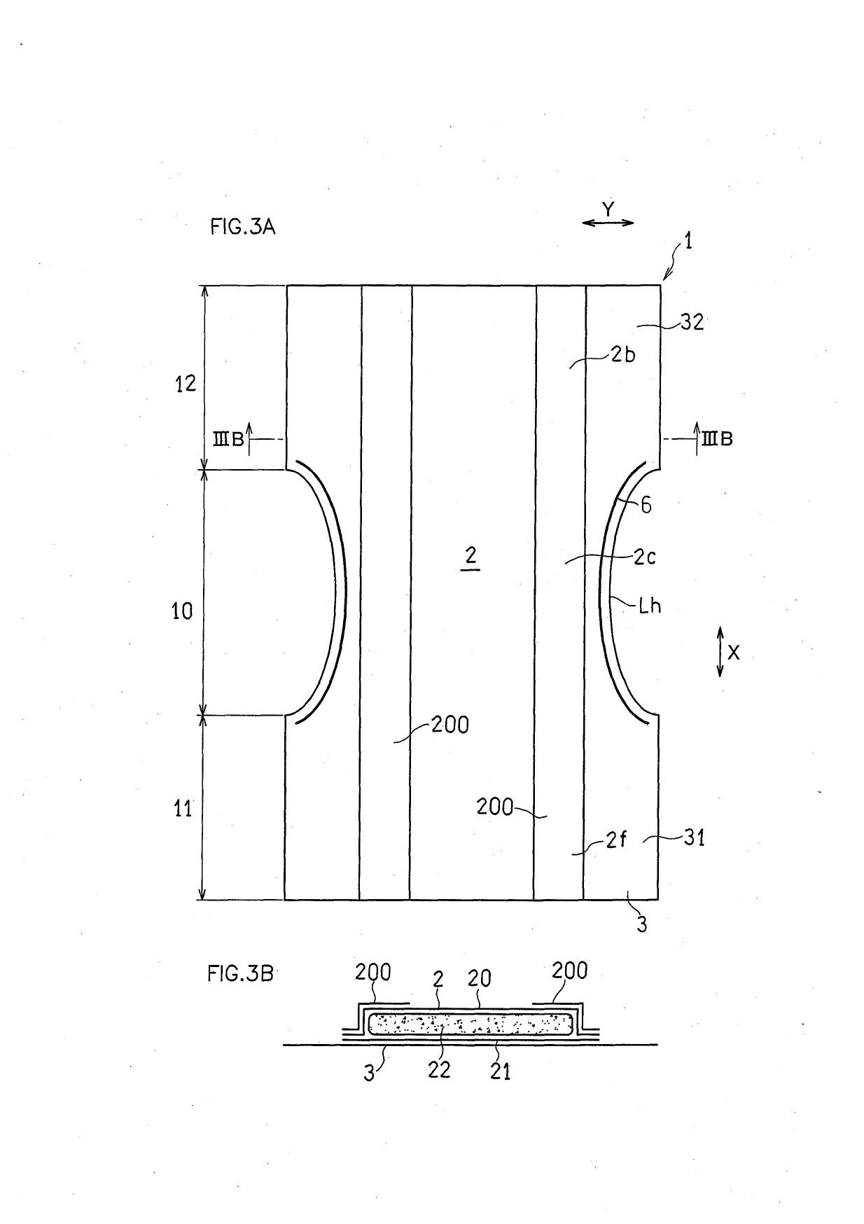

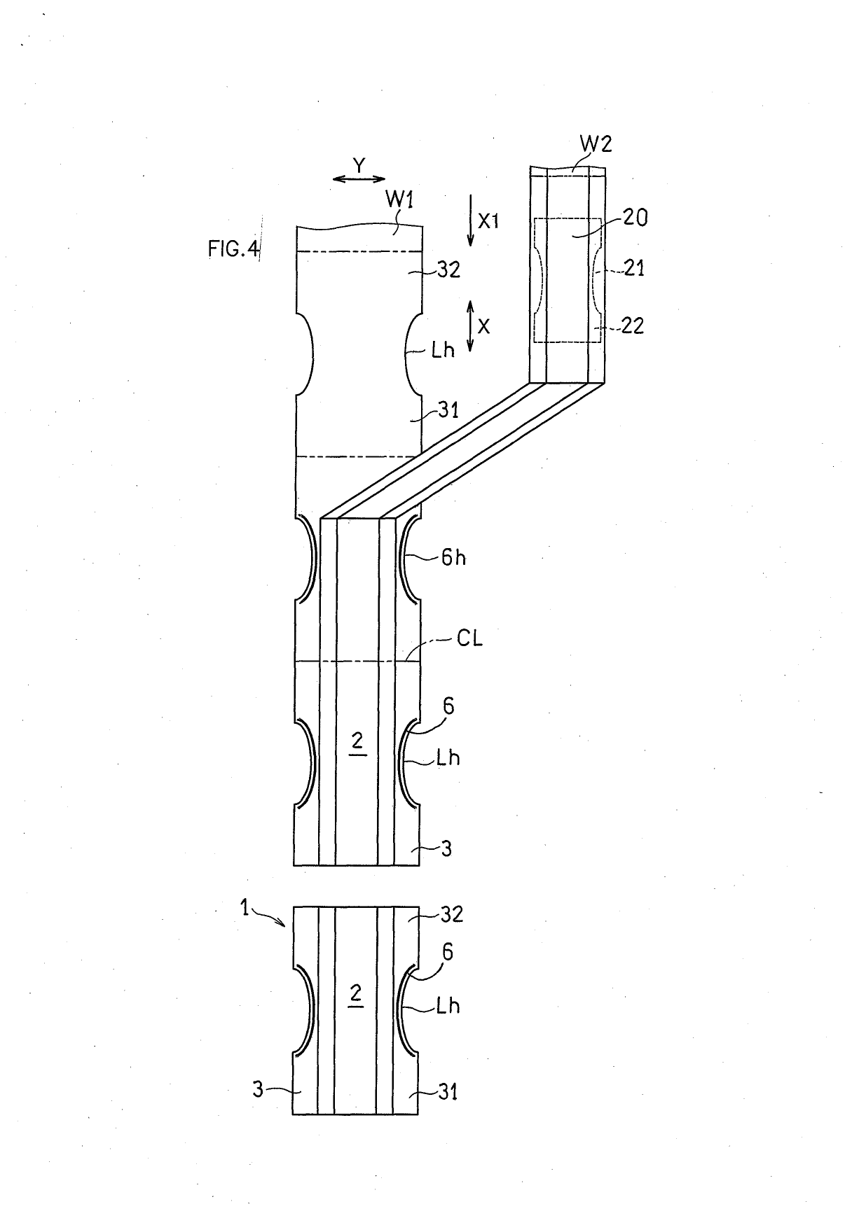

FIGS. 3A to 4 show Embodiment 2.

In Embodiment 2, the absorbent body 2 of FIG. 3A is provided extending from the front end to the rear end of the external sheet 3 in the longitudinal direction X. No elastic bands 4 are provided in Embodiment 2.

In this case, as shown in FIG. 4, after the continuous laminate W2 is layered on the continuous sheet W1, the continuous sheet W1 and the continuous laminate W2 are successively severed along the cut-off line CL.

### Embodiment 3

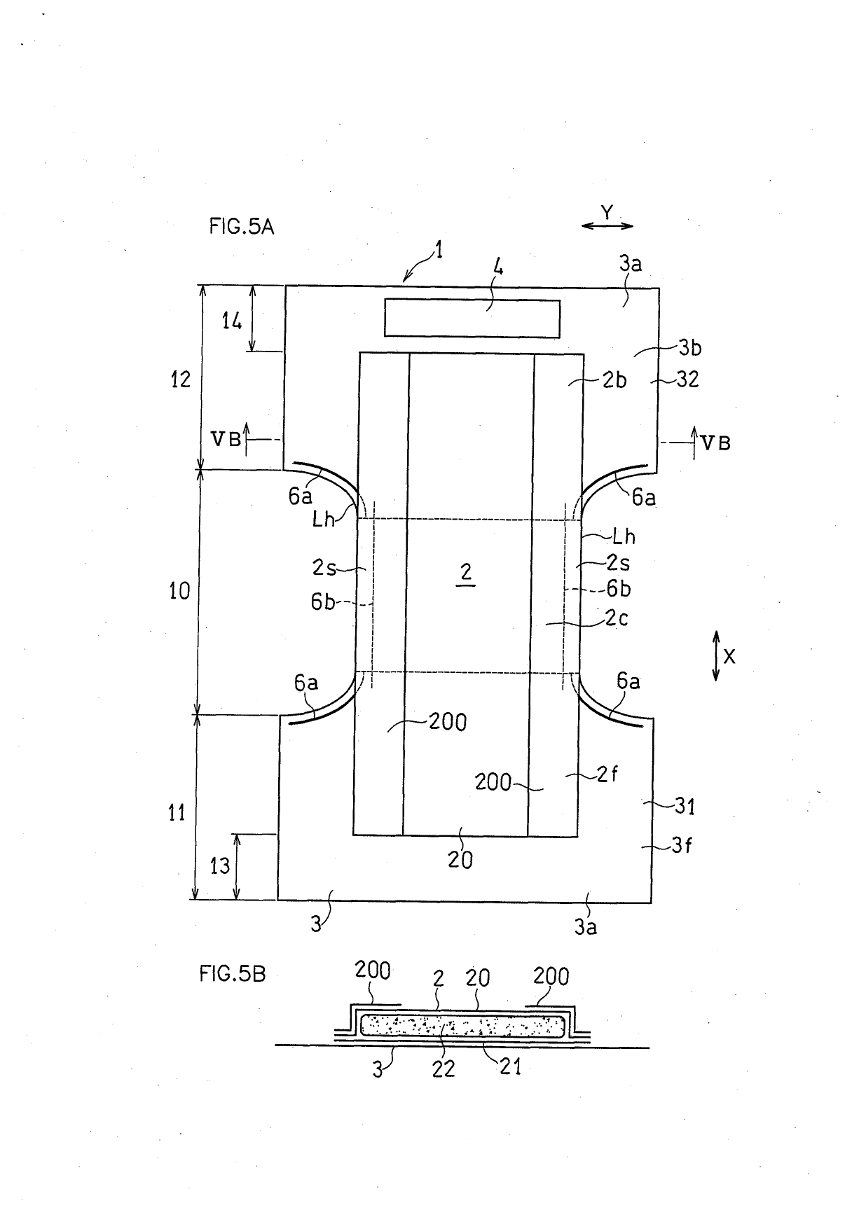

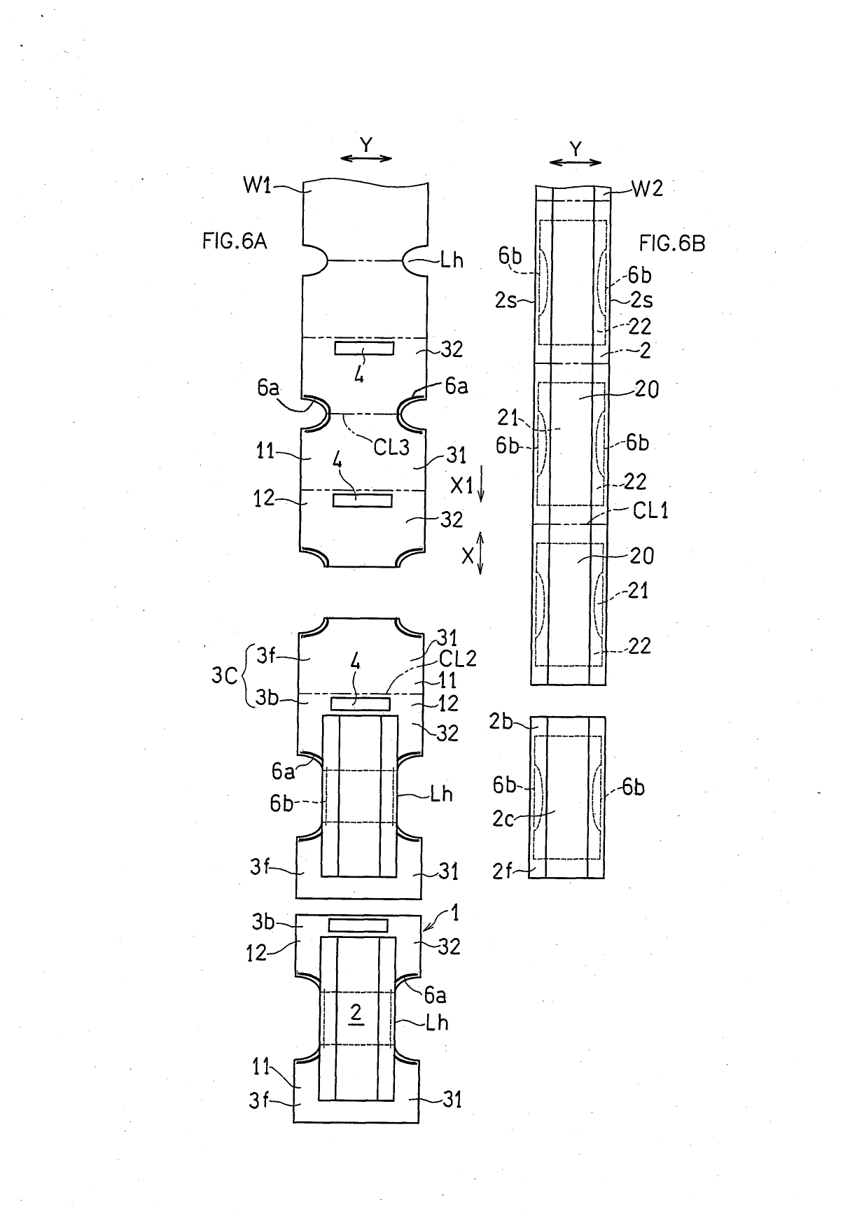

FIGS. 5A to 6B show Embodiment 3. In Embodiment 3 to be described below, what are different from Embodiment 1 described above will be mainly discussed.

Diaper 1:

As shown in FIG. 5A, in the diaper 1 of Embodiment 3, front and rear external sheets 3f and 3b are separate from each other in the crotch portion 10 between the front torso portion 11 and the rear torso portion 12. The front and rear external sheets 3f and 3b are separated from each other in the longitudinal direction X. The absorbent body 2 is, for example, bonded with an adhesive and fastened to the pair of the front torso portion 11 and the rear torso portion 12 so as to span (bridge) between the front torso portion 11 and the rear torso portion 12. Thus, the absorbent body 2 covers the crotch portion 10.

The front waist portion 13 and the rear waist portion 14, where the absorbent body 2 is absent, are formed in the front torso portion 11 and the rear torso portion 12, respectively, of the external sheets 3f and 3b, which are divided in the front-rear direction. In the waist portions 13 and 14, where the absorbent body 2 is absent, the external sheets 3f and 3b cover the waist of the wearer.

The elastic band 4 for giving stretchability in the girth direction Y to the waist portion 14 of the rear external sheet 3b is placed on the rear external sheet 3b, which covers the back side of the wearer, as in Embodiment 1.

Therefore, the inner surface 3a of the front waist portion 13 of the front external sheet 3f is exposed so as to be in contact with the skin of the wearer, and the inner surface 3a of the rear external sheet 3b is exposed so as to be in contact with the skin of the wearer between the elastic band 4 of the rear waist portion 14 of the rear external sheet 3b and the absorbent body 2.

End portions of the external sheets 3f and 3b, opposing each other, are trimmed, thereby forming parts of the leg holes Lh. First elastic members 6a made of rubber threads, for example, are placed along the end portions.

On the other hand, second elastic members 6b and 6b made of rubber threads, for example, are placed along opposite side edges 2s and 2s in the girth direction Y of the absorbent body 2 corresponding to the leg holes Lh for conformity around the legs of the wearer.

Note that the worn article may be a fastener-type diaper in which fastening members made of male touch fasteners (not shown), for example, are provided at opposite end portions of the rear external sheet 3b.

The trimmed end portions of the external sheets 3f and 3b and portions of the opposite side edges 2s of the absorbent body 2 together form the leg holes Lh, and the first and second elastic members 6a and 6b are placed around the leg holes Lh.

Next, a method for manufacturing the diaper 1 will be described.

As shown in FIG. 6A, the continuous sheet W1 made of an anisotropic non-woven fabric is carried in the carrying direction X1. The continuous sheet W1 includes the external sheets 3f and 3b of FIG. 5 continuous with one another in the longitudinal direction X perpendicular to the girth direction Y. Therefore, the continuous sheet W1 is stretchable in the girth direction Y and substantially less stretchable in the carrying direction X1 perpendicular to the girth direction Y.

While the external sheets 3f and 3b and the continuous sheet W1 are carried, a tensile force is given in the longitudinal direction X and the carrying direction X1, but no tensile force is given in the girth direction Y. That is, the continuous sheet W1 is carried while being unstretched and shrunk in the girth direction Y.

Therefore, the external sheets 3f and 3b and the continuous sheet W1 are essentially unstretchable either in the carrying direction X1 (the longitudinal direction X) or the girth direction Y, thus avoiding necking.

While being carried, portions of the continuous sheet W1 are trimmed off in a die-cutting process so that the external sheets 3f and 3b are narrowed between portions of the continuous sheet W1 to be the first and second side portions 31 and 32.

Then, as in Embodiment 1, the elastic bands 4 are placed with predetermined intervals on the continuous sheet W1.

Prior to the die-cutting process, the first elastic members 6a having a curved-arc shape are placed around the trimmed portions of the continuous sheet W1 to be parts of the leg holes Lh.

Then, as shown in FIG. 6A, severed sheets 3C are cut off from the continuous sheet W1 along the third cut-off line CL3. The severed sheet 3C is to be the rear and front external sheets 3b and 3f of adjacent diapers.

That is, the severed sheet 3C includes one of the front torso portion 11 and the rear torso portion 12 to be the preceding diaper 1, and includes the other one of the front torso portion 11 and the rear torso portion 12 to be the next diaper 1 following the preceding diaper 1. That is, as shown by two-dot-chain line (the second cut-off line CL2) in FIG. 6A, in the severed sheet 3C, portions of the external sheets 3b and 3f to be end portions of worn articles have not been cut off from each other.

Then, the front and behind severed sheets 3C and 3C adjacent to each other are separated from each other with a predetermined interval therebetween in the longitudinal direction X.

Step of Cutting Off Absorbent Body 2:

On the other hand, the continuous laminate W2 is carried as shown in FIG. 6B. In the continuous laminate W2, the second elastic members 6b are placed intermittently on the opposite side portions 2s in the girth direction Y of the absorbent body 2 to be the crotch portion.

The continuous laminate W2 is successively cut, into the absorbent body 2, along the first cut-off line CL1 generally parallel to the girth direction Y at the tip portion of the continuous laminate W2 in the carrying direction X1.

Separation Step:

After the severing, the cut-off absorbent bodies 2 are separated from one another in the longitudinal direction X.

Step of Placing Absorbent Body 2:

Then, as shown in FIG. 6A, the absorbent body 2 is provided so as to span (bridge) between a pair of severed sheets 3C and 3C separated from each other. The absorbent body 2 is placed so as to span between the front torso portion 11 side and the rear torso portion 12 side of the pair of severed sheets 3C and 3C.

Step of Severing Severed Sheet 3C:

After the placement of the absorbent body 2, the severed sheet 3C is severed along the second cut-off line CL2 to be divided into the front external sheet 3f including the front torso portion 11 and the rear external sheet 3b including the rear torso portion 12, thereby producing individual diapers 1.

Note that before the placement of the absorbent body 2, the severed sheet 3C may be severed in advance along the second cut-off line CL2.

### Embodiment 4

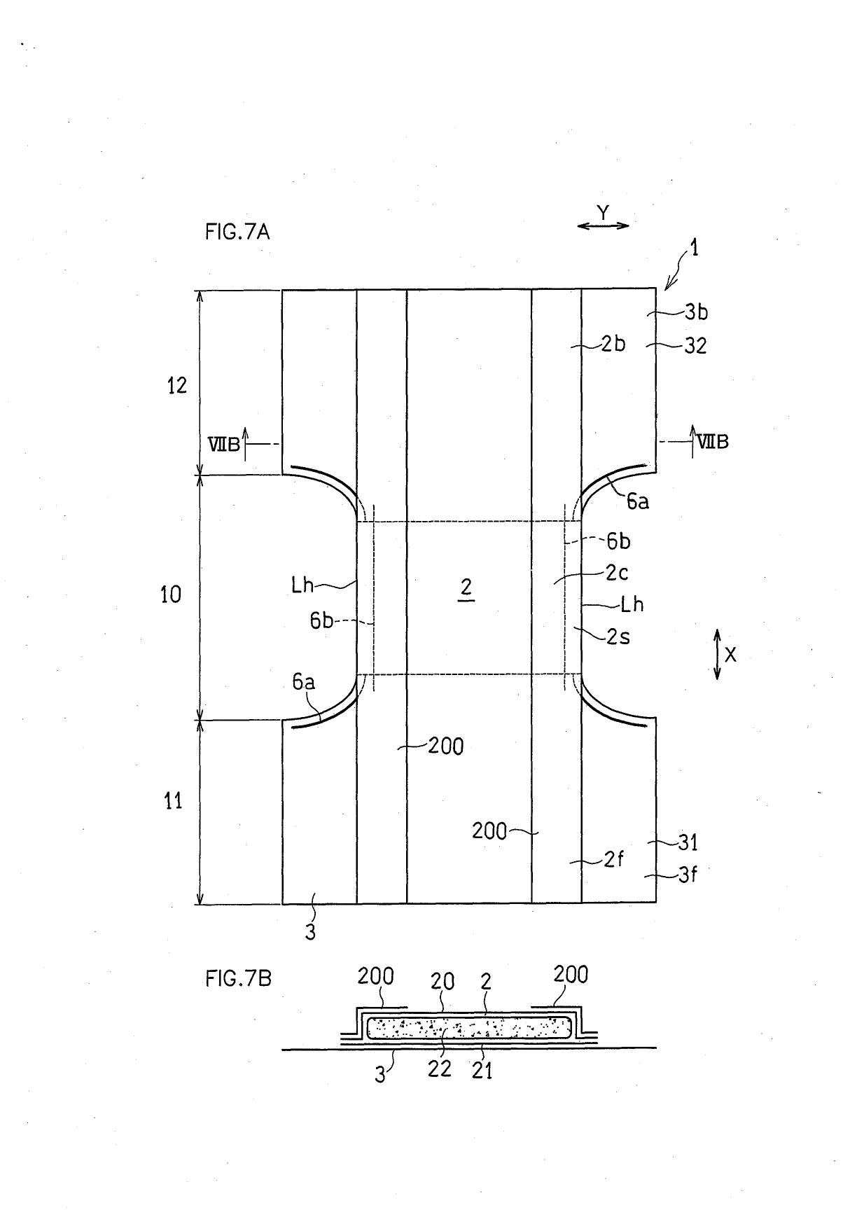

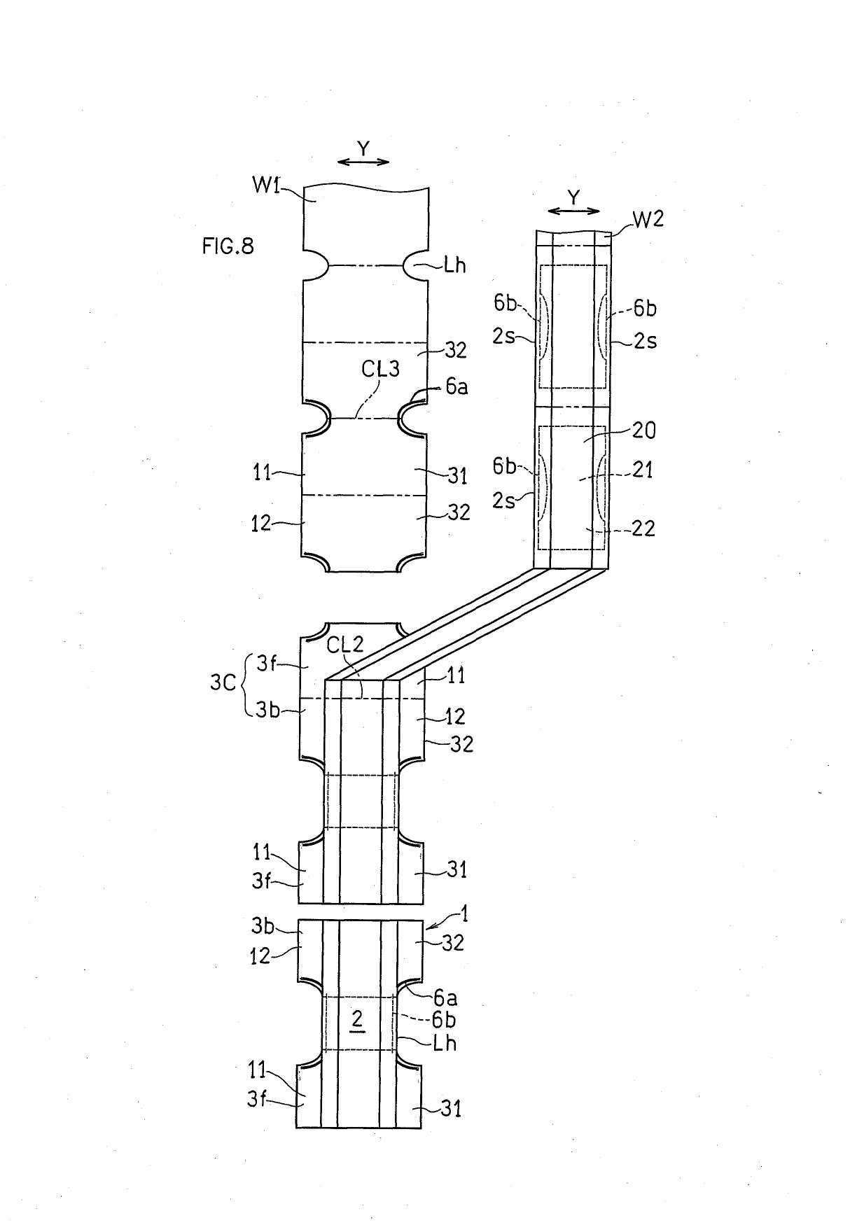

FIGS. 7A to 8 show Embodiment 4.

In Embodiment 4, the absorbent body 2 of FIG. 7A is provided extending from the front end to the rear end of the external sheet 3 in the longitudinal direction X. No elastic bands 4 are provided in Embodiment 4.

As shown in FIG. 8, in Embodiment 4, the severed sheets 3C, which are portions to be the external sheets 3, are successively cut along the third cut-off line CL3 while the first side portion 31 projecting in the girth direction Y in the front torso portion 11 of one external sheet 3f is continuous with the second side portion 32 projecting in the girth direction Y in the rear torso portion 12 of another external sheet 3b. After the severing, the cut-off severed sheets 3C are separated from each other in the longitudinal direction X.

The continuous laminate W2 to be the absorbent bodies is placed, while being continuous, on portions to be external sheets and in areas therebetween. That is, the continuous laminate W2 continuous in the longitudinal direction X is successively placed on the separated severed sheets 3C and in areas therebetween.

Then, between the first side portion 31 and the second side portion 32 of the severed sheet 3C, the severed sheet 3C and the continuous laminate W2 are successively severed along the virtual second cut-off line CL2 extending in the girth direction, thereby obtaining individual worn articles each including the absorbent body 2 placed between two severed pieces of the external sheet.

Otherwise, the configurations and the manufacturing method of the diaper 1 of Embodiment 4 are similar to those of Embodiment 3 of FIGS. 5A to 6, and therefore like elements are denoted by like reference numerals and will not be further described below.

While preferred embodiments have been described above with reference to the drawings, obvious variations and modifications will readily occur to those skilled in the art upon reading the present specification.

For example, the worn article manufactured by the present manufacturing method may be a tape-type or fastener-type diaper, or the like, in which the end portions of the front torso portion and the rear torso portion are not welded together but are fastened to each other by the fastening members.

Thus, such changes and modifications are deemed to fall within the scope of the present invention, which is defined by the appended claims.

### INDUSTRIAL APPLICABILITY

The present invention is applicable to methods for manufacturing disposable worn articles.

### REFERENCE SIGNS LIST

1: Diaper (an example of a worn article)
2: Absorbent body
3: External sheet
3C: Severed sheet
10: Crotch portion
20: Top sheet
21: Back sheet
22: Core
31: First side portion
32: Second side portion
W1: Continuous sheet
W2: Continuous laminate
X: Longitudinal direction
Y: Girth direction

1. A method for manufacturing a disposable worn article comprising an absorbent body and an external sheet, the absorbent body comprising a liquid-permeable top sheet forming a skin-contact surface to be in contact with a wearer, a liquid-impermeable back sheet forming a non-skin-contact surface on an opposite side of the skin-contact surface, and a core sandwiched between the top sheet and the back sheet for absorbing body fluid, the absorbent body including a front portion, a crotch portion and a rear portion covering a front surface of a torso of the wearer, a crotch of the wearer and a rear surface of the torso of the wearer, respectively, and the external sheet being fastened to the back sheet of the absorbent body and projecting, in a girth direction of the wearer, from the absorbent body in the front portion and the rear portion of the absorbent body, the method comprising the steps of:
carrying, in a carrying direction perpendicular to the girth direction, a continuous sheet to be the external sheet that is stretchable in the girth direction and substantially less stretchable in the carrying direction, while the continuous sheet is continuous in the carrying direction;
placing the absorbent body on the continuous sheet while the front portion, the crotch portion and the rear portion are lined up in the carrying direction; and
severing the continuous sheet along a virtual cut-off line extending in the girth direction, thereby obtaining individual worn articles each having the absorbent body placed on the external sheet.

2. A method according to claim 1, further comprising the step of:
forming, through trimming, a cut-out to be a leg hole between a first side portion projecting in the girth direction from the front portion in the external sheet and a second side portion projecting in the girth direction from the rear portion in the external sheet.

3. A method according to claim 2, further comprising the steps of:
carrying a continuous laminate to be a plurality of the absorbent bodies in the carrying direction while the continuous laminate is continuous in the carrying direction;
successively cutting off the absorbent bodies from a tip portion of the continuous laminate in the carrying direction; and
separating the cut-off absorbent bodies from one another in the carrying direction of the absorbent bodies, wherein:
in the placement step, the absorbent bodies are placed intermittently on the continuous sheet; and
the continuous sheet is severed between the adjacent absorbent bodies.

4. A method according to claim 3, wherein:
in the step of carrying the continuous sheet, no tensile force in the girth direction is given to the continuous sheet.

5. A method for manufacturing a disposable worn article comprising an absorbent body and an external sheet, the absorbent body comprising a liquid-permeable top sheet forming a skin-contact surface to be in contact with a wearer, a liquid-impermeable back sheet forming a non-skin-contact surface on an opposite side of the skin-contact surface, and a core sandwiched between the top sheet and the back sheet for absorbing body fluid, the absorbent body including a front portion, a crotch portion and a rear portion covering a front surface of a torso of the wearer, a crotch of the wearer and a rear surface of the torso of the wearer, respectively, and the external sheet being fastened to the back sheet of the absorbent body and projecting, in a girth direction of the wearer, from the absorbent body in the front portion and the rear portion of the absorbent body, the method comprising the steps of:
carrying, in a carrying direction perpendicular to the girth direction, a continuous sheet to be the external sheet of the worn article that is stretchable in the girth direction and substantially less stretchable in the carrying direction, while the continuous sheet is continuous in the carrying direction;
successively cutting off portions to be the external sheets from a tip portion of the continuous sheet in the carrying direction; and
placing the absorbent body so that the absorbent body spans between the cut-off portions to be the external sheets.

6. A method according to claim 5, wherein:
each portion to be the external sheets includes a first side portion projecting in the girth direction from the front portion of one of the external sheets and a second side portion projecting in the girth direction from the rear portion of another one of the external sheets, the second side portion being continuous with the first side portion; and
the method further comprises the step of successively dividing the portion to be the external sheets along a virtual cut-off line extending in the girth direction between the first side portion and the second side portion in the portion to be the external sheets, and obtaining individual worn articles each having the absorbent body placed between the two divided external sheets.

7. A method according to claim 6, wherein:
in the step of carrying the continuous sheet, no tensile force in the girth direction is given to the continuous sheet.

8. A method according to claim 4, wherein:
an elastic band for giving stretchability in the girth direction to a rear waist portion covering the rear surface of the torso of the wearer is placed on the rear waist portion of the external sheet, and an inner surface of the external sheet is exposed so as to be in contact with a skin of the wearer between the elastic band and the absorbent body; and
the method further comprises the step of intermittently placing a plurality of the elastic bands on the continuous sheet while no tensile force is given in the girth direction of the elastic bands.

9. A method according to claim 7, wherein:
an elastic band for giving stretchability in the girth direction to a rear waist portion covering the rear surface of the torso of the wearer is placed on the rear waist portion of the external sheet, and an inner surface of the external sheet is exposed so as to be in contact with a skin of the wearer between the elastic band and the absorbent body; and
the method further comprises the step of intermittently placing a plurality of the elastic bands on the continuous sheet while no tensile force is given in the girth direction of the elastic bands.
